(19) United States
(12) Patent Application Publication
(10) Pub. No.: US 2006/0217594 A1
(43) Pub. Date: Sep. 28, 2006

(54) Endoscopy device with removable tip

(76) Inventor: Gary W. Ferguson, Burnaby (CA)

Correspondence Address: TREXLER, BUSHNELL, GIANGIORGI,;BLACKSTONE & MARR, LTD., 105 WEST ADAMS STREET, SUITE 3600, CHICAGO, IL, 60603, US

(21) Appl. No.: 11/088,561

(22) Filed: Mar. 24, 2005

(51) Int. Cl. A61B 1/06; A61B 1/00

(52) U.S. Cl. 600/175; 600/179; 600/129

(57) ABSTRACT

The present invention provides an endoscope for in vivo imaging the cells, tissue, organs or body cavities of humans or other animals to observe and locate, diagnosis and/or treat disease. Illumination sources, image detectors, sensors may be provided alone or in combination on the removable tip allowing functional alterations or optimization for a particular procedure. Endoscope features such as an instrument channel supporting tissue sampling, suction, treatment, micro-surgery, optical computed tomography, confocal microscopy, laser or drug treatments, injections, gene-therapy, marking, implanting or other medical techniques are maintained.

### BACKGROUND OF THE INVENTION

This invention relates to the field of imaging, especially medical imaging where an animal's cells, tissue, body cavities or organelles are accessed to facilitate surgery, treatment or to diagnose disease.

Imaging is capturing electromagnetic radiation (wavelength and intensity) either reflected or emitted from an object of interest, in a manner which preserves or otherwise represents the spatial distribution of said radiation at the object. In the field of medical imaging, and more particularly endoscopy, light is utilized to illuminate body tissues and return a diagnostic or otherwise useful image. Historically, clinicians viewed white light reflectance (color) images through an ocular attached to the endoscope. More recently, with cost reductions and other computer advances, rather than viewing a tissue image through an ocular, endoscopic images are captured by an imaging device such as a video or digital camera and may be further processed for display on a monitor. Bronchoscopy serves as an example of a specific endoscopic procedure, in this instance for examining the lungs and respiratory tract. When white light is used for tissue illumination it provides visual indication of the physical structure (morphological image) of the lungs and bronchial passages. In use, clinicians may detect suspicious tissue associated with various diseases such as lung cancer by observing features in white light reflectance images such as the color and surface morphology of lung tissue and its various structures. Endoscopic procedures provide access to sample tissue/cells using scrapings, washings, brushings or biopsies. These samples would be subjected to examination by a pathologist and may be visually stained, DNA stained, tagged with bio-markers or DNA sequenced to confirm the disease status.

White light means a broad spectrum or combination of spectra in the visible range. For endoscopy, typically LEDs, lamps, lasers alone or in combination, along with optical elements such as lens, filters, filter wheels, liquid-crystal filters and multi-mirror devices, are used to provide the desired white-light illumination. It is considered advantageous for the clinician to be presented with a white-light image in real-time (at video rate), for example, to guide the endoscope through the bronchial passages. At the same time as images are displayed, images may be captured and analyzed by computer to extract various features.

Medical research indicates that cancer may be treated more effectively when detected early when lesions are smaller or when tissue is in a precancerous stage. Although changes in the physical appearance (color and morphology) of tissue using white light is useful, to accomplish more reliable and earlier detection of diseases, such as cancer, various endoscopic imaging devices have been developed which have increased sensitivity to the biological composition of tissue. Just as certain morphological changes in tissue may be associated with disease, chemical changes in cells may also be exploited for disease detection.

One such method of detecting chemical changes in tissue during an endoscopic procedure involves utilizing tissue illumination at specific wavelengths or bands of light that interact with certain chemical compounds in tissue, particularly those that are associated with diseases, such as cancer. For example, some endoscopic devices utilize light in the UV, UV/blue or IR spectrum to illuminate tissue. These wavelengths of light are selected based on their ability to stimulate certain chemicals in tissue that are associated with disease, or disease processes.

For example, when tissue is illuminated with UV, UV/blue or IR light (also called excitation), tissue may emit light. Images or spectra from these tissue emissions (fluorescence) may be captured for observation and/or analysis. Healthy and diseased tissue fluoresces differently, so the spectra of fluorescence emissions can be used as a diagnostic tool.

In addition, to assist in interpreting these fluorescence images, pseudo-colors may be assigned to help visualize the extent and location of diseased tissue. For example, the color red may be assigned to diseased tissue while healthy tissue may be displayed in green. Standardization and calibration procedures may be used to set color tones/intensities to match image characteristics from instrument to instrument or between devices from different manufacturers.

“Spectroscopy” here refers to the analysis of light according to its wavelength or frequency components. The analysis results are usually presented in the form of spectrum or spectra, which is a plot of light intensity as a function of wavelength. Reflectance spectroscopy is the analysis of reflected light from the tissue. Biological tissue is a turbid medium, which absorbs and scatters incident light. The majority of the reflected light from tissue has traveled inside the tissue and encountered absorption and scattering events, and therefore contains compositional and structural information of the tissue.

Tissue reflectance spectroscopy can be used to derive information about tissue chromophores (molecules that absorbs light strongly), e.g. hemoglobin. The ratio of oxyhemoglobin and deoxy-hemoglobin can be inferred and used to determine tissue oxygenation status, which is useful for disease detection, monitoring treatment or otherwise assessing tissue. Spectroscopy may also be used to derive information about the tissue such as cell size, size distribution and tissue density, for example.

Fluorescence spectroscopy is the analysis of fluorescence emission from tissue. Native tissue fluorophores (molecules that emit fluorescence when excited by appropriate wavelengths of light) include tyrosine, tryptophan, collagen, elastin, flavins, porphyrins, and nicotinamide adenine dinucleotide (NAD). Tissue fluorescence is very sensitive to chemical composition and chemical environment changes associated with disease transformation. Fluorescence imaging takes advantage of fluorescence intensity changes in one or more broad wavelength bands thus providing sensitive detection of suspicious tissue areas, while fluorescence spectroscopy (especially spectral shape) can be used to improve the specificity for early cancer detection.

A typical endoscope has at least one light source to provide interrogating radiation, usually white light, blue/ultraviolet light, or infrared or near-infrared light. One typical light source, for example a Xenon lamp, emits white light to produce images of the target object from light reflected or scattered from the target. Another typical light source, for example, a laser operating in a narrow band of the blue or ultraviolet region of the spectrum, emits excitation light to produce images of the target from green-shifted light fluorescing from the target. Lasers represent a narrow band light source, selected with the intention of exciting molecules. Tissue reflectance images or light signals used for image normalization may also be derived from laser light.

Light is introduced into the light guide (typically one or more fiber optics) of an endoscope and this interrogating light is directed at a target object (e.g. tissue). Interrogation light interacts with the target object providing signals related to reflectance, absorption, scattering, fluorescence, or Raman spectra. These light signals are returned the length of the endoscope, typically in a fiber bundle. When images are desired, a coherent optical fiber bundle is used. Typically, an instrument channel is also provided in the endoscope providing access to tissue for sampling tissue/cells. In addition to supporting tissue sampling devices and drawing out fluids, the instrument channel of an endoscope may provide access for micro-surgery devices, optical computed tomography, confocal microscopy, laser or drug treatments, injections, marking, implanting or other medical techniques.

Co-pending U.S. patent application Ser. No. 10/431,939, Published Patent Application No. 2004/0225222 A1 to Zeng, entitled Real-Time Contemporaneous Multi-Modal Imaging And Spectroscopy Uses Thereof, the disclosure of which is incorporated by reference, discusses means and methods to perform multi-modal endoscopic imaging where, for example, white light imaging and fluorescence imaging occur contemporaneously (at real-time video rates).

Copending U.S. patent application Ser. No. 10/453,040, Published Patent Application No. 2004/0245350 A1 to Zeng, entitled Methods and Apparatus for Fluorescence Imaging Using Multiple Excitation-Emission Pairs and Simultaneous Multi-Channel Image Detection, the disclosure of which is incorporated by reference, discusses means and methods for fluorescence imaging using multiple spectral regions.

In the past, the health-care providers viewed light or images returned by an endoscope through an ocular. Currently, it is becoming more common to position imaging devices (or spectrometers) at the position of the ocular allowing raw, processed images or spectra to be capture/displayed on a monitor.

Other advances in electronics have allowed the placement of light sources near the distal end of the endoscope. For example, by placing LEDs at the end of the endoscope, the fiber optics carrying the illumination or excitation light can be eliminated. LEDs are lower-cost, more reliable, longer-lasting, lighter in weight, more compact, and more efficient than lasers or lamps allowing for better control of illumination and consequently, imaging. Moreover, LEDs can be switched (on/off) quickly, supporting time-gated fluorescence, improved disease detection sensitivity, and increased disease detection specificity. Additionally, eliminating the interrogating radiation fiber optics confers certain benefits, including lower cost, simplified assembly, increased reliability, improved flexibility, and decreased size, for example.

Other recent innovations include placing miniature image capture devices at the distal end of the endoscope. This configuration eliminates the need for a fiber optic bundle to channel the returning radiation to a camera. Instead, the miniature image capture device sends signals to a processor such as a computer. This configuration provides an opportunity for increased resolution and consequently, improved imaging.

As endoscopes enter the body, sterilization is important, regardless of the precise type of endoscope involved, be in a bronchoscope, cardioscope, uretoscope, hysteroscope, sigmoidoscope, colonoscope, or other device. Reports of patients' contracting infections or other contagious diseases from improperly-disinfected endoscopes persist. For example, the California Department of Health Services has issued warnings to hospitals and clinics about the use of undisenfected probes in routine colon exams.

The improvements in the design of endoscopes have caused an increase in the frequency of use of endoscopes, increasing the risk of transmission of disease as instruments are used and re-used. Accordingly, a need exists for an endoscope that will reduce this risk.

Additionally, the improvements in the design of endoscopes have caused an increase in the use of endoscopes for many different medical imaging and diagnostic modes of operation. Accordingly, a need exists for a general-purpose endoscope that is easily adaptable to specific applications requiring different excitation, illumination, and sensing components.

The endoscope of the present invention meets these needs.

### BRIEF SUMMARY OF THE INVENTION

Briefly, and in accordance with the foregoing, the present invention is an endoscopy apparatus with a removable tip containing light sources and/or image capture devices, which removable tip may be changed from patient to patient. In one embodiment, the removable/disposable tip contains at least one source of interrogating radiation. In another embodiment, the removable/disposable tip contains an image capture device. In yet another embodiment, the removable/disposable tip contains both a source of interrogating radiation and an image capture device. In other embodiments, the removable/disposable tip contains apertures for surgical instruments or contains other analytical devices.

### BRIEF DESCRIPTION OF THE DRAWINGS

FIG. 1A is a diagram of an endoscopy apparatus as is known in the prior art.

FIG. 1B is a diagram of an endoscopy apparatus of the preferred embodiment of the present invention.

FIG. 2 is a cross-section of an endoscope apparatus as is known in the prior art.

FIG. 3 is a diagram of an endoscope apparatus of an embodiment of the present invention, having illumination and detection components located adjacent the distal end.

FIG. 4 is a diagram of an endoscope apparatus of an embodiment of the present invention, with illumination and detection components located on a removable tip.

FIG. 5 is a diagram of an endoscope of an embodiment of the present invention, with illumination, treatment and detection components located on a removable tip.

FIGS. 6A and 6B show two additional examples of removable tips for endoscopes of the present invention.

### DETAILED DESCRIPTION OF THE INVENTION

While preferred embodiments of the present invention are shown and described, it is envisioned that those skilled in the art may devise modifications of the present invention without departing from the spirit and scope of the invention.

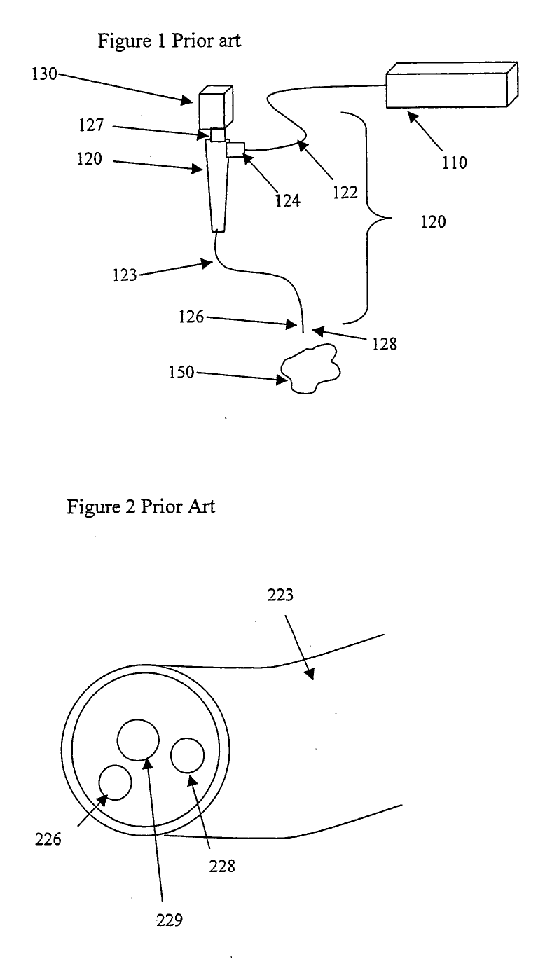

FIG. 1A shows an endoscope apparatus as is known in the prior art. Light source 110 generates interrogating radiation which can be some combination of white light for color images, narrow-band excitation light for fluorescence, or other narrow bands for spectral analysis or image normalization, or other types of light. Illumination source 110 provides light into endoscope assembly 120 via illumination fiber bundle 122 at junction 124. Illumination exits the distal end 123 of the endoscope 120, which in this example contains two fiber bundles 126, 128. Fiber bundle 126 directs illumination/excitation light to target tissue 150. The interrogating radiation is incident on a target 150, which will reflect, scatter, or fluoresce the interrogating radiation depending on what type of light was used to illuminate the target. Fiber bundle 128 directs returning light for spectroscopy or imaging to sensor 130 mounted adjacent the ocular 127 of endoscope assembly 120.

FIG. 2 shows a cross-section of the distal portion 223 of the endoscope assembly of FIG. 1A. Fiber bundle 226 directs interrogating radiation to the target and fiber bundle 228 carries the spectral and imaging radiation back to a detector such as a camera or spectrometer. The endoscope may also provide instrument channels such as biopsy channel 229.

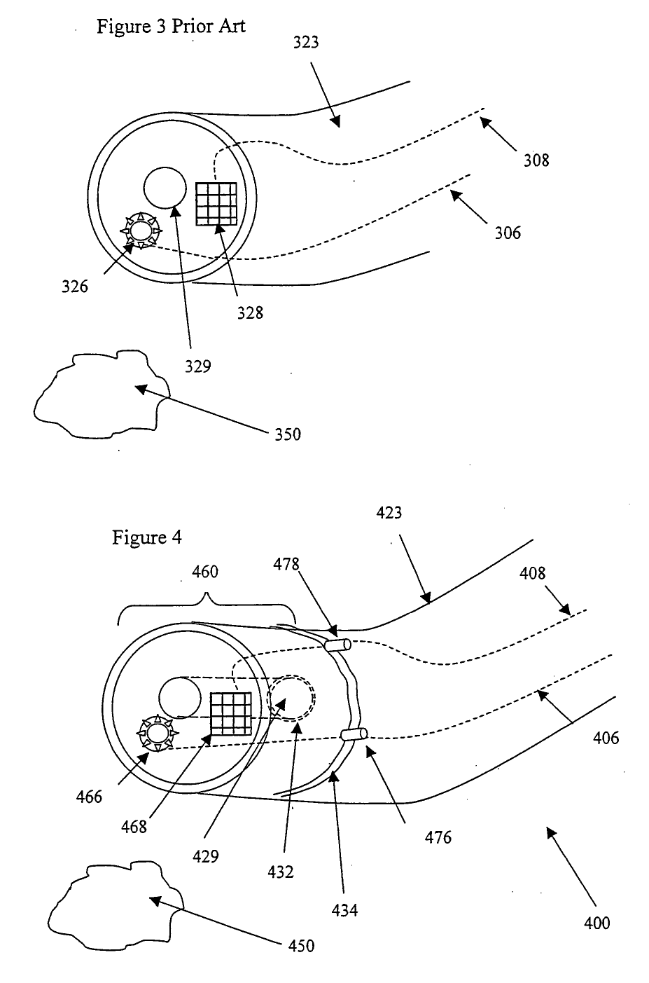

FIG. 3 shows a cross-section of the distal portion 323 of another endoscope assembly of the prior art, with illumination and detection components. Illumination source(s) 326 may include one or more LED(s) to generate the desired interrogating radiation onto the target tissue 350. Power/control of the illumination source 326 is provided through wires 306. A detector circuit 328 captures radiation such as spectral and imaging radiation with power provided by, and data communicated for image or spectral analysis through wires 308. The endoscope may also provide instrument channels such as biopsy channel 329.

The present invention simplifies the arrangement shown in FIG. 1A. As shown in FIG. 1B, the endoscope assembly 160 of the preferred embodiment of the present invention has a tube 168 that is insertable into a patient, either through a body cavity or a surgical incision. Tube 168 has, at its distal end, a removable tip assembly, as will be described in connection with FIGS. 3 through 6B. The removable tip assembly, as will be described, contains some combination of a light source, a sensor, and possibly surgical or analytical devices. Interrogating radiation is therefore produced within the removable tip assembly for irradiation of a target 150, and/or returning radiation is detected within the removable tip assembly and signals corresponding to that returning radiation are transmitted to detector 164, which can be a color monitor, a spectrograph, or other detector. Tube 168 therefore contains a conduit for transmission of power to the components, as well as for transmission of returning signals to detector 164. In the preferred embodiment, the signals are transmitted by conduit 170, which can be a metal wire, a light guide such as an optic fiber, or other means of transmitting digital or analog signals. The signal transmission portion of conduit 170 and tube 167 can be continuous.

In another embodiment, the removable tip contains a transmitter. In this embodiment, signals corresponding to the returning radiation are transmitted from the tip by radio signals, eliminating the need for a signal transmission component to tube 168 and conduit 170. In yet another embodiment, a power source such as disposable batteries are located in the tip assembly itself, eliminating the need for a power transmission component to tube 168.

FIG. 4 shows a cross-section of the distal portion 423 of an endoscope assembly 400 of an embodiment of the present invention, with illumination and detection components located on a removable tip assembly 460.

The removable tip assembly 460 seals in a fluid-tight manner to the distal portion 423 of the endoscope assembly 400. Sealing gasket 434 preferably is used to provide a seal.

Illumination source(s) 466, located in removable tip assembly 460, may include one or more LED(s) to generate the desired interrogating radiation onto the target tissue 450. Power/control of the illumination source is introduced by wires 406 which are connected to the removable tip assembly 460 by plug connector 476. A detector circuit 468 located on removable tip assembly 460 captures radiation such as spectral and imaging radiation from interrogated tissue 450. Wires 408, which connect at plug 476, transmit power and data for image or spectral analysis. The endoscope may also provide instrument channels such as biopsy channel 429 provided in the removable tip assembly 460.

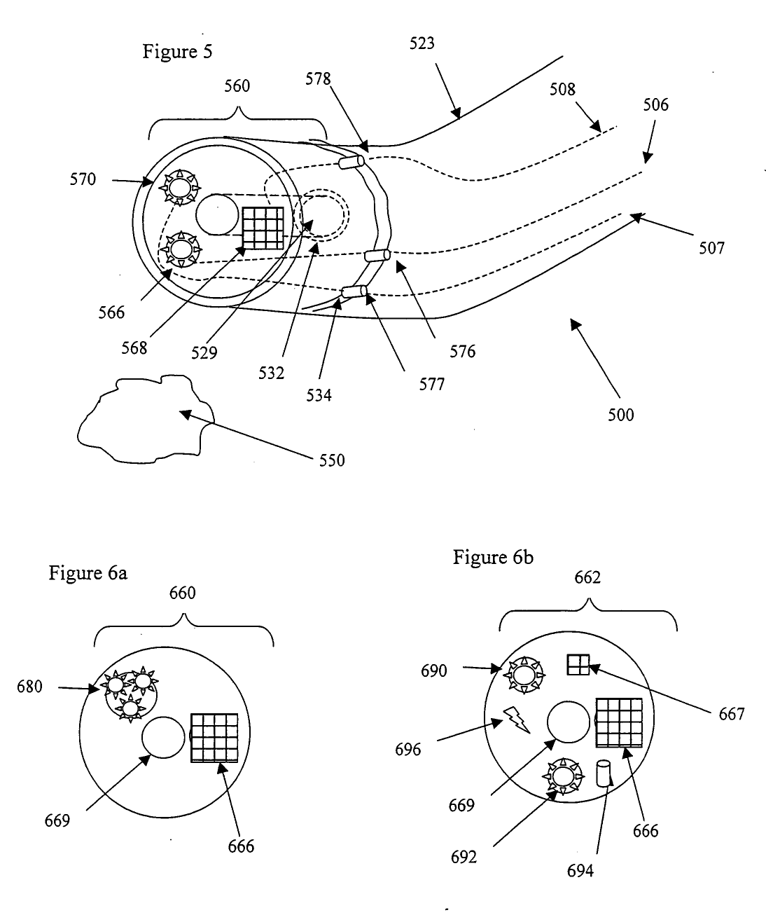

FIG. 5 shows a cross-section of the distal portion 523 of another embodiment of an endoscope assembly 500 of the present invention, with illumination and detection components located on a removable tip assembly 560. As with the embodiment noted in connection with FIG. 4, removable tip assembly 560 seals in a fluid-tight manner to the distal portion 523 of the endoscope assembly 500. Sealing gasket 534 preferably is used to provide a tight seal.

Illumination source(s) 566, located in removable tip assembly 560, may include one or more LED(s) to generate the desired interrogating radiation onto the target tissue 550. Power/control of the illumination source 566 is introduced by wires 506 which are connected to the tip assembly 560 by plug connector 576. A detector circuit 568 located on removable tip assembly 560 captures radiation such as spectral and imaging radiation from interrogated tissue 550. Power and data communicated for image or spectral analysis are provided by wires 508 which connect at plug 576. The endoscope 500 may also provide instrument channels such as biopsy channel 529 in removable tip assembly 560.

In this embodiment, a treatment light source 570 is also located on the removable tip assembly 560, receiving power and control via wires 507 which junction at plug connector 577. This embodiment illustrates that desired components typically utilized for endoscopic procedures may be located on the removable tip assembly 560, allowing a single endoscopic assembly 500 to be customized or otherwise be adapted to an application.

Removable tip assemblies 460, 560 may be attached to endoscope assemblies 400, 500, respectively, via press fit, threads, interference fit, screws, connectors, or other secure temporary types of attachment. Lenses, filters, optical coatings, and other optical components may comprise part of the removable tip assemblies 460, 560. Additional passages for suction, treatment, radiation etc. may be conveyed through removable tip assemblies 460, 560.

Instrument channels 429, 529, in addition to introducing biopsy or other tissue sampling devices, provide access for micro-surgery devices, releasing nano-devices, optical computed tomography, confocal microscopy, laser or drug treatments, gene-therapy, injections, marking, implanting or other medical techniques.

FIGS. 6A and 6B show other embodiments of the present invention. FIG. 6A shows a removable tip assembly 660 for basic white light illumination and imaging. FIG. 6B shows a removable tip assembly 662 with additional light sources for fluorescence and an additional detector for spectral measurements.

FIG. 6A is the end view of a removable tip assembly 660 for an endoscope assembly such as those discussed in association with FIGS. 4 and 5. In this instance, removable tip assembly 660 contains LED 680 providing broadband illumination with three LEDs, which, for example, could be red, green and blue. Also shown are instrument channel 669 and detector circuit 666. Other analytical devices could also be mounted on the removable tip such as DNA-chips, micro-machines, chemical analyzers etc.

FIG. 6B shows another removable tip assembly 662 with a single broad source LED 690 for illumination, a near-infrared LED 692 to stimulate fluorescence and a detector 667. Other combinations of illumination sources, excitation sources, image capture devices, and other analytical devices are possible within the present invention.

Removable tip assembly 662 also contains an internal power source 694 and a radio transmitter 696. Internal power source 694 is preferably a disposable battery, but could be a rechargeable battery or other power source. Radio transmitter 696 relays the signals from detector 667, which may detect returning broadband images and/or returning fluorescence images for transmission to a monitor or camera. Radio transmitter 696 could relay returning radiation from detector 667 to a spectrograph for spectroscopy analysis.

In all embodiments described, the removable tip assembly can be de-attached from the endoscope assembly for cleaning, replacement, repair, or disposal. A removable tip assembly containing inexpensive components can be removed and disposed of. The user can open a sterilized package containing a replacement tip assembly and attach it to the endoscope for the next procedure. Removable tip assemblies with expensive components can be removed and separately sterilized with radiation, ethylene oxide, steam, or other suitable sterilization protocol. A removable tip assembly with a broken part can be easily transported to a repair shop for repair, such as replacement of an LED, without transporting the entire apparatus.

A further advantage of a removable tip assembly is the possibility of using a separate sterilization protocol for the removable tip assembly, than used for the rest of the endoscope. Normally, the entire endoscope must be sterilized between procedures on different patients. Some sterilization protocols, such as some heat sterilization procedures, can be harmful to electronic components. Accordingly, a removable tip assembly allows the use of one sterilization protocol for the main part of the endoscope, such as an autoclave, and a second sterilization protocol, such as ethylene oxide, for the tip assembly. Moreover, because the removable tip assembly is quite small in relation to the rest of the endoscope, a more aggressive or a more expensive sterilization protocol may be used for the removable tip assembly, while a different sterilization protocol is used for the rest of the endoscope.

Accordingly, endoscopic tip replacement provides a means to update, customize and better adapt endoscopes to particular applications. Complex or expensive components may be removed from one device and re-used on another device. As new illumination sources, detectors, sensors, analytical micro-devices, and treatment options become available, it may be more cost effective to locate such components on a removable tip obviating the need to replace basic assemblies or replicate expensive components. As component costs decline it may become preferable to keep several sterilized tips on hand which may be recycled or could be cleaned, sterilized with radiation, ethylene oxide, steam, or other suitable sterilization protocol for re-use. Similarly, advances in miniaturization, microsurgery and other medical techniques may be advanced by these innovations.

While preferred embodiments of the present invention are shown and described, it is envisioned that those skilled in the art may devise various modifications of the present invention without departing from the spirit and scope.

I claim:

1. An endoscope assembly comprising an endoscope and a removable tip, said tip comprising at least one of a radio transmitter, a power source, and an illumination light source.

2. (canceled)

3. The endoscope assembly of claim 1, wherein said removable tip further comprises at least one of an image detector, an instrument channel, and an analytical device.

4. The endoscope assembly of claim 1, wherein said removable tip further comprises at least one image detector.

5. (canceled)

6. The endoscope assembly of claim 4 wherein said removable tip further comprises at least one of an instrument channel and an analytical device.

7. The endoscope assembly of claim 1, wherein said removable tip further comprises at least one instrument channel.

8. The endoscope assembly of claim 7, wherein said removable tip further comprises at least one analytical device.

9. The endoscope assembly of claim 1, wherein said removable tip is disposable.

10. The endoscope assembly of claim 1, wherein said removable tip is sterilizable.

11. The endoscope assembly of claim 1, wherein said removable tip is repairable.

12. The endoscope assembly of claim 1, wherein said removable tip further comprises at least one of a lens, a filter, and an optical coating.

13. (canceled)

14. An endoscope comprising:
a tube having a proximal end and a distal end for inserting said distal end into a body cavity or surgical incision of a patient and comprising a first conduit for transmitting power and a second conduit for transmitting signals;
a removable tip comprising an illumination source and an image detector;
a connector to removably attach said tip to said tube, to connect said first conduit to said illumination source and said image detector, and to connect said second conduit to said image detector.

15. The endoscope of claim 14, wherein said removable tip further comprises at least one of a lens, a filter, and an optical coating.

16. The endoscope of claim 14, wherein sad removable tip further comprises at least one of an instrument channel and an analytical device.

17. The endoscope of claim 14, wherein said removable tip is disposable.

18. The endoscope of claim 14, wherein said removable tip is sterilizable.

19. The endoscope of claim 14, wherein said removable tip is repairable.

20. The endoscope of claim 14, wherein said removable tip further comprises an internal power source.

21. The endoscope of claim 14, wherein said removable tip further comprises a radio transmitter.

22. The endoscope assembly of claim 1, wherein said light source comprises a light-emitting diode.

23. The endoscope assembly of claim 1, wherein said light source comprises a broadband light-emitting diode and a narrow-band light-emitting diode.

24. The endoscope assembly of claim 1, wherein said power source comprises at least one of a disposable battery and a rechargeable battery.

25. The endoscope assembly of claim 1, further comprising a connector.

26. The endoscope assembly of claim 25, wherein said connector comprises at least one of a press fit and an interference fit.

27. The endoscope assembly of claim 25, wherein said connector comprises a fluid-tight sealing gasket.

28. The endoscope assembly of claim 14, wherein said illumination source comprises a light-emitting diode.

29. The endoscope assembly of claim 14, wherein said illumination source comprises a broadband light-emitting diode and a narrow-band light-emitting diode.

30. The endoscope assembly of claim 14, wherein said power source comprises at least one of a disposable battery and a rechargeable battery.

31. The endoscope assembly of claim 14, wherein said connector comprises at least one of a press fit and an interference fit.

32. The endoscope assembly of claim 14, wherein said connector comprises a fluid-tight sealing gasket.

33. A removable tip for an endoscope, comprising:
a light source producing interrogating radiation for illumination of a target;
a detector circuit for receiving radiation produced by the target in response to the interrogating radiation;
a radio transmitter for relaying signals from said detector to at least one of a monitor, a camera, and a spectrograph;
a power source coupled to said light source, said detector, and said radio transmitter; and
means for removably attaching said light source, said detector, said radio transmitter, and said power source to an endoscope.

34. The removable tip of claim 33, further comprising at least one of a lens, a filter, and an optical coating.

35. The removable tip of claim 33, further comprising at least one of an instrument channel and an analytical device.

36. The removable tip of claim 33, wherein said removable tip is disposable.

37. The removable tip of claim 33, wherein said removable tip is sterilizable.

38. The removable tip of claim 33, wherein said removable tip is repairable.

39. The removable tip of claim 33, wherein said light source comprises a light-emitting diode.

40. The removable tip of claim 33, wherein said light source comprises a broadband light-emitting diode and a narrow-band light-emitting diode.

41. The removable tip of claim 40, wherein said narrow-band light-emitting diode comprises a near-infrared light-emitting diode.

42. The removable tip of claim 40, wherein said broadband light-emitting diode comprises a red light-emitting diode, a green light-emitting diode, and a blue light-emitting diode.

43. The removable tip of claim 33, wherein said detector circuit detects at least one of spectral radiation and imaging radiation.

44. The removable tip of claim 33, wherein said detector circuit comprises a spectral radiation detector and an imaging radiation detector.

45. The removable tip of claim 33, wherein said power source is at least one of a disposable battery and a rechargeable battery.

46. The removable tip of claim 33, further comprising an analytical device comprising at least one of a DNA-chip, a micro-machine, and a chemical analyzer.

47. The removable tip of claim 33, further comprising a connector.

48. The removable tip of claim 47, wherein said connector comprises at least one of a press fit and an interference fit.

49. The removable tip of claim 47, wherein said connector comprises a fluid-tight sealing gasket.
